(19) United States
(12) Patent Application Publication
(10) Pub. No.: US 2013/0296882 A1
(43) Pub. Date: Nov. 7, 2013

(54) APPARATUS FOR SURGERY

(71) Applicant: Young Jae Kim, (US)

(72) Inventors: Young Jae Kim, Seoul (KR); Nathanael KIM, Los Angeles, CA (US)

(21) Appl. No.: 13/854,241

(22) Filed: Apr. 1, 2013

Related U.S. Application Data
(60) Provisional application No. 61/618,528, filed on Mar. 30, 2012.

(51) Int. Cl. A61B 19/00

(52) U.S. Cl. CPC A61B 19/2203
USPC 606/130

(57) ABSTRACT

A surgical manipulator comprising: a master module configured to be controlled by a user; a slave module configured to move according to movement of the master module; and a transmission module comprising gears and configured to operably connect the master module and the slave module.

### CROSS REFERENCE TO RELATED APPLICATIONS

Pursuant to 35 U.S.C. §119(e), this application claims the benefit of Provisional Application No. 61/618,528 filed on Mar. 30, 2012, the contents of which are hereby incorporated by reference herein in their entirety.

### FIELD

The present disclosure generally relates to a method and an apparatus for enhancing performance of minimally invasive surgery. In particular, the present disclosure relates to a mechanical manipulator for manipulating surgical instruments used in the minimally invasive or endoscopic surgery. The present disclosure more particularly relates to a surgical instrument which provides a high degree of dexterity and precision, allowing easy control of surgical instruments and improving performance of minimally invasive surgical procedures.

### DESCRIPTION OF THE RELATED ART

In a conventional operation, an incision is made on a body of a patient such that a surgical instrument and sometimes a hand of a surgeon can be placed inside the body. Therefore, in the conventional operation, a surgeon must stand closely to the patient so that the surgeon has direct access to the inside the body of the patient. The surgeon must hold the surgical instrument in his or her hand to manipulate the surgical instrument. Therefore, in a complicated surgery, the surgeon needs to perform the operation in a standing posture for several hours, and thus, the surgeon gets very tired during the surgery.

Endoscopic surgery or minimally invasive surgery can be performed with a small incision compared to the incision made in the conventional surgery. Since the endoscopic surgery is characterized by accessing a human body through one or more incisions and ports, a surgeon's hand does not need to be put into the patient's body. While a single port may be sufficient to perform the surgery, more than one ports may be necessary in some occasions to accommodate a number of instruments inside the body.

One example of the endoscopic surgery is laparoscopic surgery which is minimally invasive surgery inside the abdominal cavity. Usually, a video camera or laparoscope and several thin instruments are used for the laparoscopic surgery. The incision may be up to half an inch or about 12 mm and the port, which is usually a plastic tube, is placed through the incision such that the camera and surgical instruments are introduced through the port, allowing access to the inside of a patient's body. A surgeon performs a surgical procedure, observing images captured by the video camera via an image viewer, such as a monitor, a head mounted display, or video glasses, during the laparoscopic surgery.

For example, the surgical instruments or endo effectors used for the laparoscopic surgery include clamps, graspers, scissors, staplers, and needle holders. Typically, while these surgical instruments used for the laparoscopic surgery are similar to those used in conventional open surgery, the working end of each instrument is separated from its handle by a long extension tube, for example, of about 12 inches or about 300 mm in length such that movement of the endo effector positioned on the distal ends of the surgical instrument and located at the surgical site can be controlled via the handle from outside the patient's body.

During the laparoscopic surgical procedure, a surgeon manipulates the surgical instruments that have been passed through the port or cannula sleeve to an internal surgical site from outside the patient's body. The surgical instruments can be slid in and out through the port and rotated. Further, the endo effectors on the distal ends of the surgical instruments can be actuated from outside the patient's body while the endo effectors and the surgical site are monitored by means of a laparoscopic camera and a video monitor.

Therefore, during a laparoscopic surgical procedure, a surgeon's hands may not be used to directly hold and move the surgical instruments. Rather, the surgeon's hands are actually moved some distance away from the surgical site and the surgical instruments are controlled via a handle or controller that is directly coupled to the surgical instrument. This procedure also requires the surgeon to stand very close to the patient as in the conventional surgery. For example, the combined length of the surgical instrument coupled to the controller may be about 30 cm and not more than 50 cm. A learning curve for manipulating the surgical instrument coupled to the controller may be long. Similar to the conventional surgery, a surgery performed using this type of surgical instrument coupled to the controller may also require long standing by the surgeon, and the surgeon performing the surgery may also get tired after a few hours of surgery.

In other words, in response to input received via the controller, the end effectors of the surgical instruments that are inserted through the port into the internal surgical site of the patient's body are moved. Various surgical effectors that can be connected to the surgical instruments are available.

Further, when several instruments need to be inserted into the body together, more than one person may be required to perform the surgery such that each person holds or operate each instrument since one person cannot hold or handle all the instruments. For example, a first person may hold an instrument having a needle, a second person may hold an instrument having an endoscope or video camera, and a third person may hold an instrument having a grasper that is used to hold an internal organ.

Moreover, robotic systems have become popular for performing laparoscopic procedures and the robotic systems may solve the problems of the portable hand-held manual surgical manipulator described above. These robotic systems allow more complex movements of surgical instruments in a limited space due to additional degrees of freedom available to the surgeon. Therefore, the learning curve may be reduced when robotic systems are used in laparoscopic surgery compared to conventional manual laparoscopic instruments described above. However, such robotic systems are very costly (more than $ 1 million), and thus, not many hospitals or doctors can afford such expensive robotic systems. Further, such robotic systems, which are motorized, electrically driven and often computer-assisted, are very complex and lack force feedback to the surgeon.

Portable mechanical surgical instruments or handheld manipulators, such as the surgical instrument that is coupled to the controller, are more affordable than the robotic systems previously discussed. In general, such portable mechanical surgical instruments consist of two hand-guided mechanical manipulators which may allow multiple degrees of freedom based on a deflectable and rotatable tip of the instruments. Therefore, the structure of the portable mechanical surgical instruments may be much simpler than the structure of robotic systems. Further, one disadvantage of the portable mechanical surgical instruments is that it may be more tiring for a surgeon to perform surgery with the portable mechanical surgical instruments than to perform a surgery using the robotic systems because the surgeon performs the surgery in a standing position when using the portable mechanical surgical instruments while the surgeon may sit in a chair in front of a control station when using the robotic systems. On the other hand, in general, the robotic systems require using only surgical instruments specifically manufactured to be used with the robotic system and such surgical instruments that are compatible with the robotic systems are very costly, and thus, the cost of maintenance is very high. Therefore, a surgical system that is affordable and allows precise work without getting the surgeon tired is necessary.

### SUMMARY

The present disclosure provides a endoscopic/laparoscopic surgical system that is affordable and easy to use.

In accordance with one embodiment of the present invention, a surgical manipulator comprising: a master module configured to be controlled by a user; a slave module configured to move according to movement of the master module; and a transmission module comprising gears and configured to operably connect the master module and the slave module, wherein the master module comprises: an upper arm with a proximal end connected to a first end portion of the transmission module; a lower arm with a proximal end connected to a distal end of the upper arm of the master module; and a handle portion connected to a distal end of the lower arm and configured to receive a user input, wherein the slave module comprises: an upper arm with a proximal end connected to a second end portion of the transmission module; a lower arm with a proximal end connected to a distal end of the upper arm of the slave module; and an adapter connected to a distal end of the lower arm of the slave module and configured to receive a surgical instrument, wherein the transmission module is configured to transmit a mechanical force to the slave module in order to cause movement of at least the upper arm of the slave module, the lower arm of the slave module, the adapter, or the surgical instrument attached to the adaptor; and wherein the mechanical force is generated in response to the user input received via the handle portion.

In accordance with another embodiment of the present invention, the transmission module is further configured to: rotate the upper arm of the master module and the upper arm of the slave module synchronously; raise or lower the upper arm of the slave module synchronously in response to raising or lowering of the upper arm of the master module; move the lower arm of the slave module back and forth synchronously in response to back and forth movement of the lower arm of the master module; raise or lower the upper and lower arms of the slave module synchronously in response to raising or lowering the upper and lower arms of the master module; rotate the adapter of the slave module synchronously in response to rotation of the handle portion of the master module in order to rotate the lower arms of the master module and the slave module; and move the adapter back and forth in response to back and forth movement of the handle portion.

In accordance with another embodiment of the present invention, a range of rotation of the upper arm of the master module and the upper arm of the slave module is between approximately ±46 degrees.

In accordance with another embodiment of the present invention, a range of a variable angle between the upper arm of the master module and the lower arm of the master module is approximately ±50 degrees.

In accordance with another embodiment of the present invention, a range of motion for raising or lowering the upper arm of the master module is approximately ±30 degrees.

In accordance with another embodiment of the present invention, a range of rotation of the lower arm of the master module is approximately ±170 degrees.

In accordance with another embodiment of the present invention, the handle portion is rotatable about a vertical axis and movable back and forth or up and down.

In accordance with another embodiment of the present invention, the adapter is detachable from the slave module and replaceable with another type of adapter.

In accordance with another embodiment of the present invention, the surgical manipulator further comprises a plurality of adapters, wherein each of the plurality of adapters is configured to receive a specific type of surgical instrument that is compatible with the corresponding adapter.

In accordance with another embodiment of the present invention, the plurality of adapters are configured to receive various commercially available surgical instruments that are manufactured by different manufacturers.

In accordance with another embodiment of the present invention, the transmission module further comprises a pulley.

In accordance with another embodiment of the present invention, a length of the upper arm of the master module is approximately 10 to 15 inches; a length of the lower arm of the master module is approximately 10 to 15 inches; a length of the upper arm of the slave module is approximately 10 to 15 inches; and a length of the lower arm of the slave module is approximately 10 to 15 inches.

In accordance with another embodiment of the present invention, an angle between the upper arm and the lower arm of the master module is within a range of approximately 1 to approximately 359 degrees according to a position of the lower arm of the master module; and an angle between the upper arm and the lower arm of the slave module is within a range of approximately 1 to approximately 359 degrees according to the position of the lower arm of the master module.

In accordance with another embodiment of the present invention, the angle between the upper arm and the lower arm of the master module and the angle between the upper arm and the lower arm of the slave module are substantially the same when the upper arm and the lower arm of the master module are positioned to form an angle of approximately 90 degrees.

In accordance with another embodiment of the present invention, the angle between the upper arm and the lower arm of the slave module is greater than the angle between the upper arm and the lower arm of the master module when the upper arm and the lower arm of the master module are positioned to form an angle of less than approximately 90 degrees.

In accordance with another embodiment of the present invention, the angle between the upper arm and the lower arm of the slave module is less than the angle between the upper arm and the lower arm of the master module when the upper arm and the lower arm of the master module are positioned to form an angle of greater than approximately 90 degrees.

In accordance with another embodiment of the present invention, the upper arm of the master module and the upper arm of the slave module are rotated about an X-axis that is a lengthwise axis of the transmission module.

In accordance with another embodiment of the present invention, the lower arm of the slave module and the lower arm of the master module are moved back and forth along the X-axis.

In accordance with another embodiment of the present invention, at least an angle between the lower arm and the upper arm of the slave module or an angle between the lower arm and the upper arm of the master module is changed when the lower arm of the slave module and the lower arm of the master module are moved back and forth synchronously.

In accordance with another embodiment of the present invention, the transmission module requires no electrical power.

In accordance with another embodiment of the present invention, each of the master module and the slave module has seven degrees of freedom.

In accordance with another embodiment of the present invention, the surgical manipulator further comprises a multi-channel breaker configured to stop motion of the master module and the slave module at a desired position.

In accordance with another embodiment of the present invention, the surgical manipulator further comprises an actuator configured to power the breaker.

In accordance with another embodiment of the present invention, the handle portion comprises a wheel configured to cause a tip of the surgical instrument to at least rotate, bend, or grab; and the wheel is rotatable at least up and down or left and right.

In accordance with another embodiment of the present invention, the surgical manipulator further comprises a counterweight or spring balancer configured to balance weights provided in the master module and the slave module.

In accordance with another embodiment of the present invention, the adapter comprises a guide pin configured to align and stabilize attachment of the surgical instrument to the adapter.

In accordance with another embodiment of the present invention, the surgical manipulator further comprises multiple master modules and multiple slave modules, wherein each of the multiple slave modules is configured to be positioned at various heights.

In accordance with another embodiment of the present invention, the adapter has three degrees of freedom.

In accordance with another embodiment of the present invention, the upper arm and the lower arm of the master module are connected via a first joint; and the upper arm and the lower arm of the slave module are connected via a second joint.

In accordance with another embodiment of the present invention, each of the first joint and the second joint comprises a brake structure configured to hold the corresponding first joint and second joint at a desired position.

In accordance with another embodiment of the present invention, a surgical manipulator comprising: a master module configured to be controlled by a user; a slave module configured to move according to movement of the master module; a transmission module comprising gears and configured to operably connect the master module and the slave module; and a multi-channel breaker configured to stop motion of the master module and the slave module at a desired position, wherein the master module comprises: an upper arm with a proximal end connected to a first end portion of the transmission module; a lower arm with a proximal end connected to a distal end of the upper arm of the master module; and a handle portion connected to a distal end of the lower arm and configured to receive a user input, wherein the slave module comprises: an upper arm with a proximal end connected to a second end portion of the transmission module; a lower arm with a proximal end connected to a distal end of the upper arm of the slave module; and an adapter connected to a distal end of the lower arm of the slave module and configured to receive a surgical instrument.

In accordance with another embodiment of the present invention, A surgical manipulator comprising: a plurality of master modules configured to be controlled by a user; a plurality of slave modules configured to move according to movement of the respectively corresponding plurality of master modules and to be positioned at various heights; a plurality of transmission modules comprising gears and configured to operably connect the plurality of master modules and the respectively corresponding plurality of slave modules; and wherein each of the plurality of master modules comprises: an upper arm with a proximal end connected to a first end portion of the transmission module; a lower arm with a proximal end connected to a distal end of the upper arm of the master module; and a handle portion connected to a distal end of the lower arm and configured to receive a user input, wherein each of the plurality of slave modules comprises: an upper arm with a proximal end connected to a second end portion of the transmission module; a lower arm with a proximal end connected to a distal end of the upper arm of the slave module; an an adapter connected to a distal end of the lower arm of the slave module and configured to receive a surgical instrument.

These and other embodiments will also become readily apparent to those skilled in the art from the following detailed description of the embodiments having reference to the attached figures, the invention not being limited to any particular embodiment disclose.

### BRIEF DESCRIPTION OF THE DRAWINGS

FIG. 1A shows a mechanical manipulator according to an example embodiment of the invention.

FIG. 1B shows a mechanical manipulator held by a supporting stand according to another example embodiment of the invention.

FIG. 1C shows a mechanical manipulator installed on a table according to yet another example embodiment of the invention.

FIG. 1D shows a mechanical manipulator with a brake system according to yet another example embodiment of the invention.

FIG. 2 shows a schematic kinematic diagram of a mechanical manipulator according to an example embodiment of the invention.

FIGS. 3A-3C show a handle assembly of the mechanical manipulator according to an example embodiment of the invention.

FIG. 4 shows a slave module of the mechanical manipulator shown in FIG. 1A according to an example embodiment of the invention.

FIG. 5 shows a transmission assembly of the mechanical manipulator according to an example embodiment of the invention.

FIG. 6 shows an adapter receiving a surgical instrument according to an example embodiment of the invention.

FIG. 7A-7B show a cross section view of the adapter shown in FIG. 6.

FIG. 8 shows a brake used at a joint according to an example embodiment of the invention.

### DETAILED DESCRIPTION OF EXAMPLE EMBODIMENTS

In the following detailed description, reference is made to the accompanying drawing figures which form a part hereof, and which show by way of illustration example embodiments of the invention. It is to be understood by those of ordinary skill in this technological field that other embodiments may be utilized, and structural, electrical, as well as procedural changes may be made without departing from the scope of the example embodiments of the invention described herein. Wherever possible, the same reference numbers will be used throughout the drawings to refer to the same or similar parts.

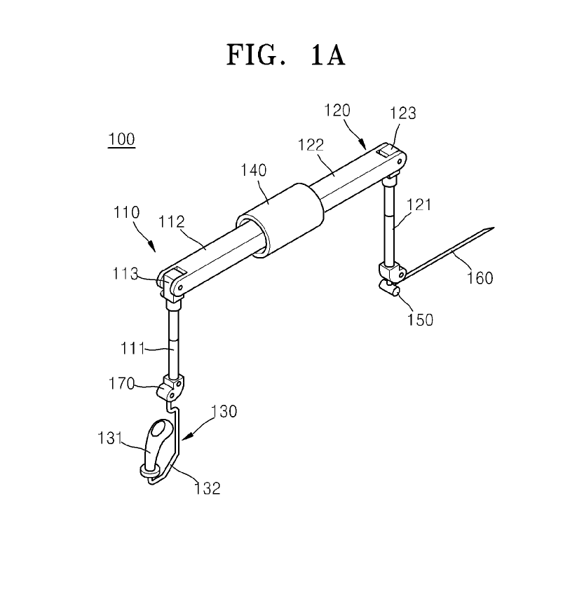

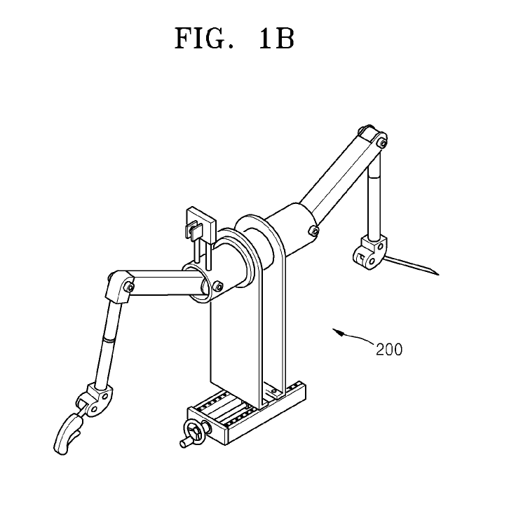

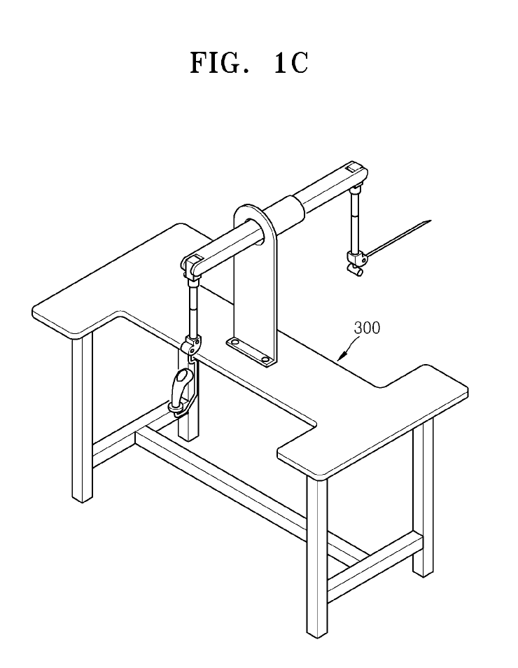

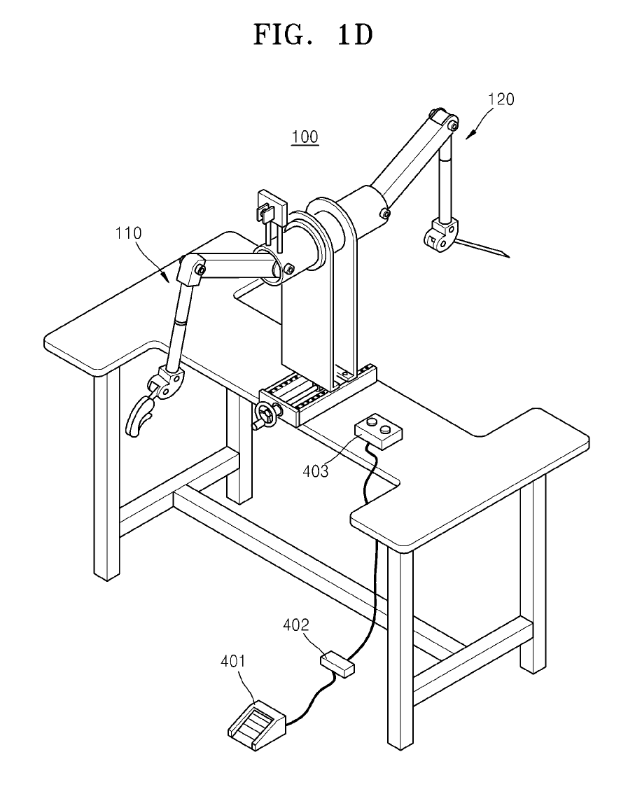

Referring to FIG. 1A, an apparatus 100 for manipulating an end-effector or surgical instrument 160 includes a master module 110 and a slave module 120. The master module 110 and the slave module 120 may be operatively connected by a transmission assembly 140 and the slave module 120 moves according to movement of the master module 110. In other words, the transmission assembly 140 transfers the motion of the master module 110 to the slave module 120 in order to manipulate a surgical instrument 160 attached to the slave module 120. Preferably, the apparatus 100 is not motorized and the surgical instrument 160 is manipulated mechanically in response to user's control which provides mechanical energy, i.e., according to the motion and position a controller controlled by the user. For example, the transmission module 140 includes at least a plurality of chains or a plurality of cables to transfer mechanical power generated from the master module 110 to the slave module 120.

Conventional endoscopic instruments have a limited amount of freedom for positioning. The ergonomic quality of conventional laparoscopic instruments is also poor and a surgeon often operates the instruments in an uncomfortable posture with extreme wrist positions. In one example embodiment of the present invention, sizes and dimensions of the master module 110 and the slave module 120 of the apparatus 100 are different and a balanced counterweight or spring may be required for smooth operation without inducing fatigue to a user.

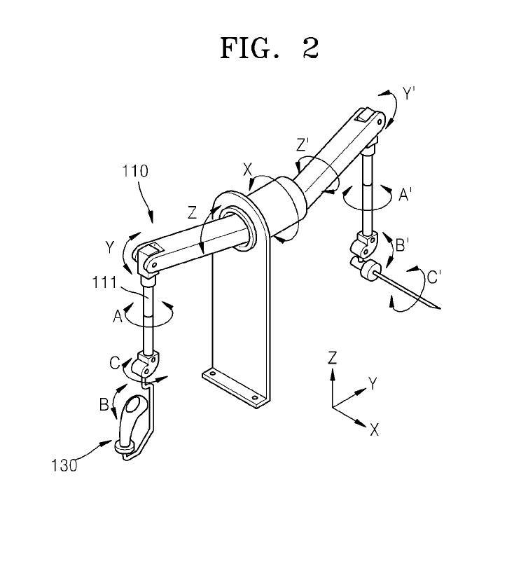

For example, the master module 110 includes a handle assembly 130, a wrist assembly 170, a lower arm 111 and an upper arm 112, as shown in FIG. 1A. The lower arm 111 and the upper arm 112 may be connected via a joint 113 and each of the lower arm 111 and the upper arm 112 has several degrees of freedom: X, Y, Z, azimuth (A), wrist elevation (B), and wrist rotation (C), as shown in FIG. 2.

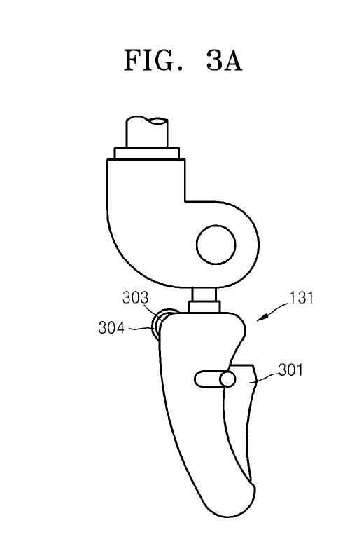

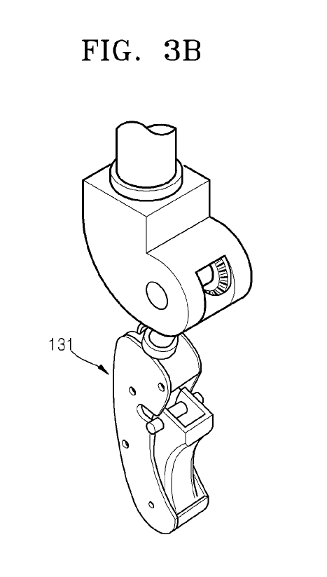

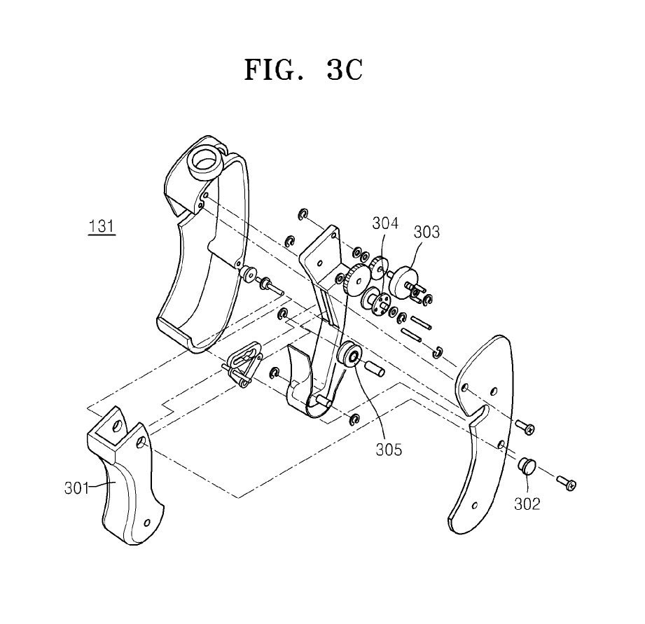

The handle assembly 130 may include an operating handle 131. Referring to FIGS. 3A and 3B, the operating handle 131 may include at least one trigger 301 and a lock switch/button 302. The operating handle 131, which is designed ergonomically, may further include a wheel type roller 303. For example, the trigger 301 and the wheel type roller 303 may be positioned on opposite sides of the operating handle 131 and the wheel type roller 303 may be controlled by a thumb of a user's hand holding the operating handle 131. Further, the handle assembly 130 may be connected to the wrist assembly 170 directly or via an adapter 132, as shown in FIG. 1A. For example, the operating handle 131 may be a type of joystick which can be moved in various directions as controlled by a user. In other words, the operating handle 131 may be raised, pushed, pulled, or rotated clockwise or counterclockwise.

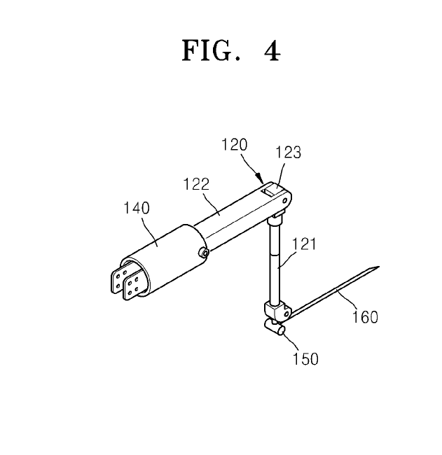

Referring to FIG. 4, the slave module 120 includes a tool assembly to which an end-effector or surgical instrument 160 and/or an adapter 150 for a laparoscopic tool is detachably coupled. The slave module 120 may include a lower arm 121 and an upper arm 122 connected via a joint 123 and the lower arm 121 may be rotated in response to input received at the master module 110. Lengths of the lower arm 121 and the upper arm 122 may be similar. For example, the surgical instrument 160 may be coupled to or decoupled from the lower arm 121 of the slave module 120 by a snap-fit mechanism. The surgical instrument 160 may be connected to the lower arm 121 of the slave module 120 via a manual snap-fit adapter 150. For example, the surgical instrument 160 may have a screw structure and may be coupled to the adapter 150 by turning the screw structure. The surgical instrument 160 that is connected to the lower arm 121 of the salve module 120 may be disposable or reusable.

In one example embodiment of the present invention, the surgical instrument 160 may include a tip at a distal end and the tip may be rotated and/or deflected in response to input received at the master module 110, for example via the operating hand assembly 130 (not shown in the drawing). Further, the tip of the surgical instrument 160 may also perform an grabbing action in response to the input received via the hand assembly 130. For example, the input may be received via the wheel type roller 303 of the hand assembly 130 to deflect the tip of the surgical instrument 160.

The apparatus 100 may also be referred to as a mechanical tele-manipulator and used for laparoscopic surgery. According to an example embodiment of the present invention, the apparatus 100 may be held by a supporting stand 200 as shown in FIG. 1B. According to another example embodiment, the apparatus 100 may be attached to a table fixture 300 as shown in FIG. 1C and the table fixture may be height adjustable. A monitor screen required for endoscopic surgery may be installed at the table fixture 300 or near the table fixture 300. The apparatus 100 is configured such that an operator can manipulate the apparatus 100 in a sitting position while sitting on a chair. For example, the operator or surgeon may be seated at least more than about 1 meter away from the patient who is placed on an operation table and control the surgical instrument 160 via the operating handle 131.

The apparatus 100 may be used to hold and manipulate a laparoscope and various types of surgical instruments 160 that are used during the surgery. In general, the laparoscope and surgical instrument 160 attached to the apparatus 100 accesses the human body through a small incision and port. In one example embodiment, a plurality of apparatus 100 may be used together and multiple arms may be provided by the plurality of apparatus 100, each arm having a different type of surgical instrument 160.

In another example embodiment, the plurality of apparatus 100 may be installed on a single table fixture 300 and the multiple arms may have different lengths or the length of at least the upper arms may be adjusted as necessary, for example, depending on a type of the surgery or the patient's posture. For example, four apparatuses 100 may be used together, two apparatuses 100 being used to control surgical instruments, one apparatus 100 being used to hold an endoscope, and the other apparatus 100 being used to handle a retractor for holing an internal organ. Since the lower arm 121 of the slave module 120 in each of the four apparatuses 100 can be fixedly positioned, a single surgeon may handle each apparatus 100 at a time.

When four apparatuses 100 are installed on a single table fixture 300, the apparatuses may be arranged in series in a single row or in tandem. For example, the table fixture 300 may have more than one layer, for example two layers, and two apparatuses 100 may be installed on a first layer and the other two apparatuses 100 may be installed on a second layer of the table fixture 300. Alternatively, the table fixture 300 may have a curved surface and when the four apparatuses 100 are installed on the table fixture 300 in series, at least one or each of the four apparatuses 100 may be installed at a different height.

The apparatus 100 is designed for easy coupling/decoupling of the surgical instrument 160 and conveys an operator's dexterity to the surgical instrument 160. The surgical instrument 160, for example, may include graspers, scissors, retractor, radio frequency (RF) or ultrasonic cutter, needle holders, and the like, and are interchangeable such that an appropriate end effector may be attached to the apparatus 100 depending on the surgical task.

Referring to FIG. 2, the master module 110 is configured to generate motions in various directions and have nine degrees of freedom (DOF) including X (left/right), Y (forward/backward), Z (up/down), A (azimuth), B (wrist elevation), C (wrist rotation) and gripping (not shown in the drawing). For example, in response to a user's control input, the master module 110 may generate X-motion of about ±46° or more on a wall mounting, Y-motion that allows arms to swing back and forth at about ±50° or more from a neutral position, and Z-motion of about ±30° with respect to a horizontal axis. Further, the ergonomic operating handle assembly 130 of the master module 110 may allow azimuth rotation of a lower arm 111 at about ±170° or more, wrist elevation with a motion of between about 45° or more in an upwards direction and about −135° or less in a downwards direction with respect to a horizontal axis, and wrist rotation with a motion of about ±210° or more. Referring to FIG. 1D, according to yet another example embodiment, the apparatus 100 may be operably coupled with a brake system 401, 402, 403 for X, Y, and Z motions of the master module 110 and the slave module 120, counter weight balancers for X and Y motions, and stoppers for X and Y motions.

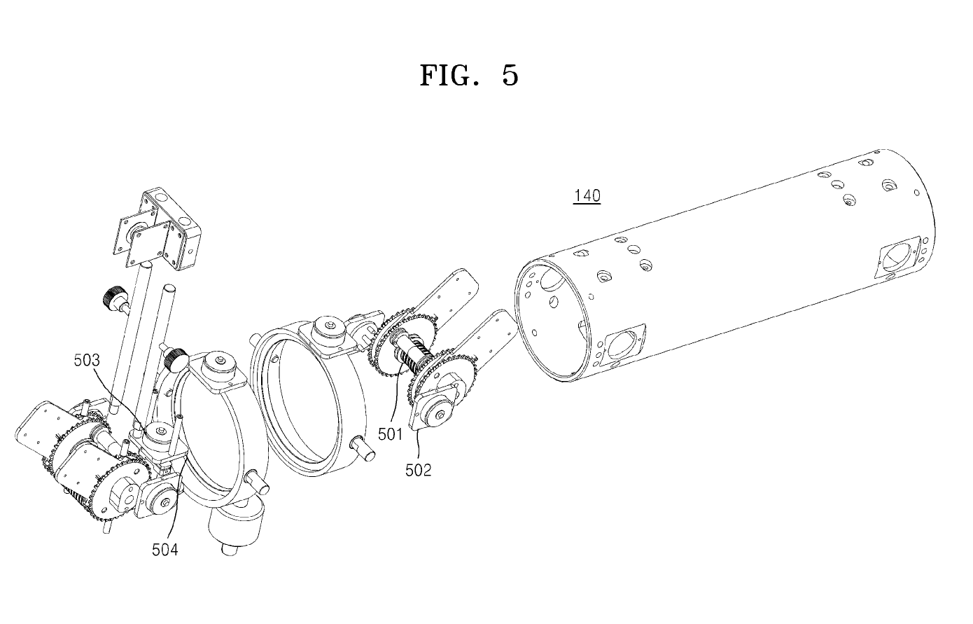

In one example embodiment, the transmission assembly 140 includes sprockets and chains for controlling the Z motion. For example, the transmission assembly 140 may include four sprockets and chains. The transmission assembly 140 may further include pulleys 501 and tendon wires (not shown) for controlling the wrist and azimuth motions. For example, the transmission assembly may 140 include seven or more pulleys and tendon wires. An example embodiment of the transmission assembly 140 is shown in FIG. 5.

The wrist assembly includes two bevel gears for wrist elevation and rotation. The wrist assembly further includes a pulley and tendon wire for opening/closing a gripper (not shown in the drawing).

The handle assembly 130 is connected to the lower arm 111 of the master module 110, as shown in FIG. 1A. In one example embodiment, the operating handle 131 may be connected to the lower arm 111 via the adapter 132, as shown in FIG. 1A. The operating handle 131 may be a pistol-grip style and may also have a three channel grabber lock switch. The operating handle 131 may further have a wheel-style control knob 303 used to control the azimuth or wrist motions, as shown in FIG. 3A. In one embodiment, the handle assembly 130 includes pulleys 304, 305 used for tool swing and gripping, as shown in FIG. 3C. The handle assembly 130 may further include a locking mechanism such that a position of the surgical instrument 160 may be locked by activating the locking mechanism via the lock switch/button 302, as shown in FIGS. 3A-3C.

The master module 110 may further include a joint 113 connecting the lower arm 111 and the upper arm 112, as shown in FIG. 1A. The handle assembly 130 is operatively coupled to the lower arm 111. The lower arm 111 can be moved back and forth by pushing or pulling the handle assembly 130. Further, the upper arm 112 may be operatively coupled to the lower arm 111 at one end and to the transmission assembly 140 at the other end. The transmission assembly 140 may be operated by a direct link mechanism. The transmission assembly 140 may be operatively coupled with a brake system such as a solenoid 502, 503, 504, as shown in FIG. 5.

The apparatus 100 may also include a plurality of breaker structures that are configured to hold a selected joint separately or independently. Activation of the plurality of breaker structures causes each arm to stay locked in a specific position. For example, each arm may have three joints on which solenoids are located for the breakers. Further, the breakers may be turned on and off via input received via a pedal switch 401 which is controlled with a foot, as shown in FIG. 1D. According to example embodiment, the pedal switch 401 may be used as a multi-purpose switch to control not only the breakers, but also to control other functions such as function of the endoscope and electronic instruments used during the surgery.

Furthermore, the breakers may be associated with indicators 403 which indicate the operation status, as shown in FIG. 1D. For example, the indicators 403 may include at least one light which is turned on when the breaker is activated. Alternatively, the indicators 403 may include a plurality of lights and different light will be lit depending on the operation status, i.e., activated or deactivated breaker. Counter weights may be adjusted with a counter weight balancer or spring balancer.

The slave module 120 may also have seven degrees of freedom for its motion that is controlled according to the motion of the master module 110. The surgical instrument 160 is detachably attached to the slave module 120 such that movement of the surgical instrument 160 can be controlled from the master module 110.

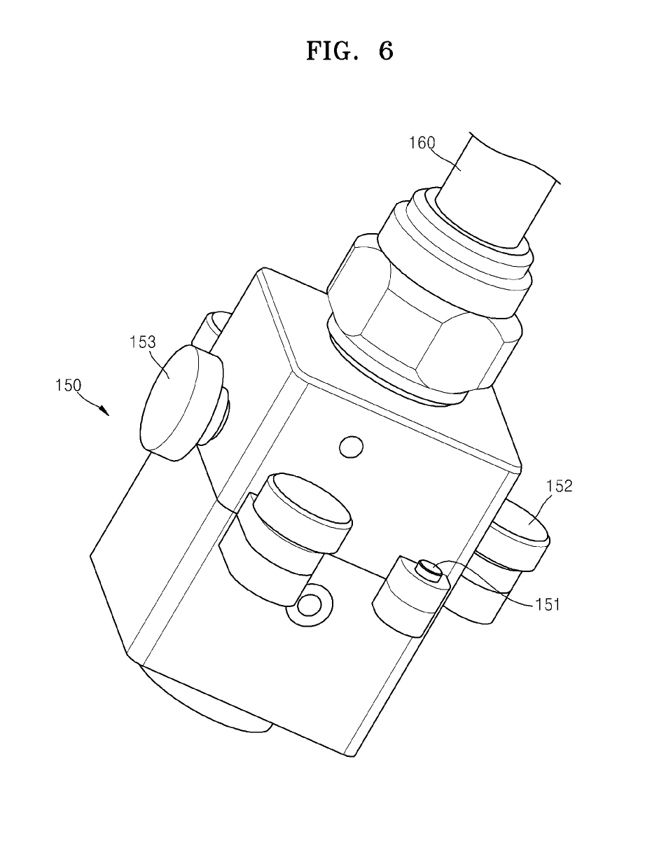

Referring to FIGS. 4 and 6, the surgical instrument or end effector 160 may be attached to the adapter 150 of the slave module 120. For example, the adapter 150 may have three degrees of freedom to allow tip rotation, tip deflection, and tip open/close functions.

Referring to FIG. 6, the adapter 150 may be a pin guided assembly and may also have knobs for assembly of the adapter 150. For example, the adapter 150 may have two guiding pins 151, three knobs 152 with screws, and a tip rotation knob 153.

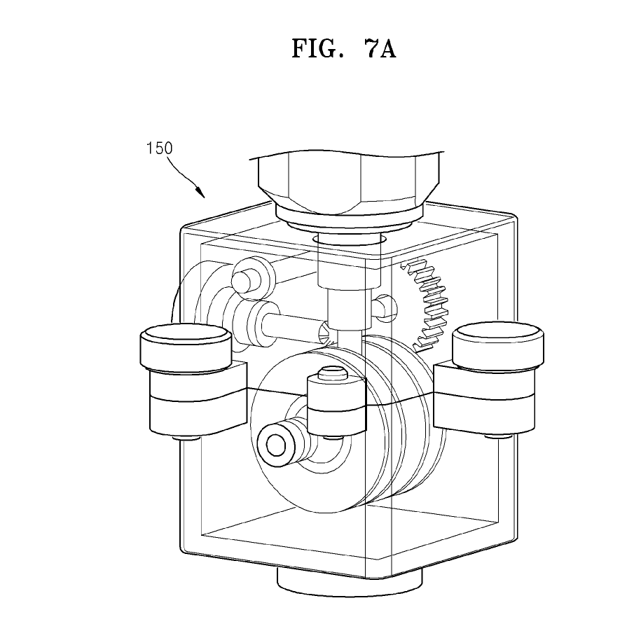

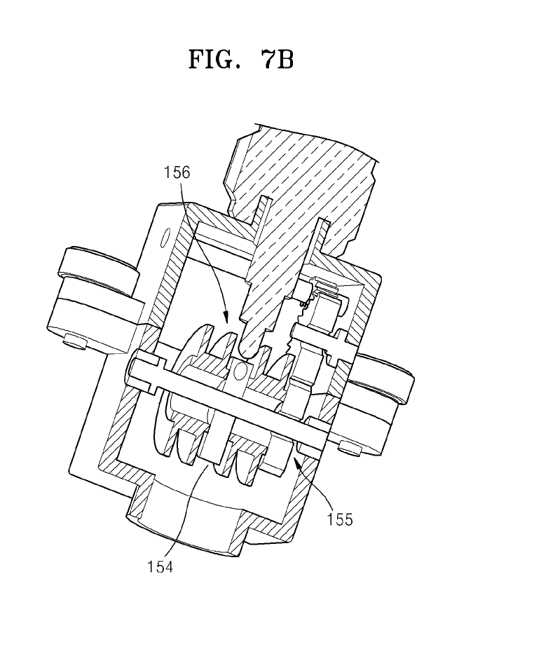

Referring to FIG. 7, the adapter 150 may further have a cam 154 for opening/closing the tip and a gear/pulley 155 for tip deflection. The tip rotation knob 153 may be equipped with a bevel gear 156.

In one example embodiment, the adapter 150 may be customized to receive any type of surgical instruments or end effectors 160 including conventional surgical instruments. Thus, different types of adapters 150 may be attached to the lower arm 121 of the slave module 120 depending on the surgical instrument that will be attached to the apparatus 100.

In another example embodiment, the surgical tools may be modified or partially disassembled to be attached to the adapter 150. For example, the surgical tools may be tools that are available commercially or used widely and a handle portion of a surgical tool may be detached from the surgical tool such that the remaining portion or tool portion can be attached to the adapter 150.

In particular, surgical tools that are normally held directly by the surgeon may be attached to the apparatus 100 via the adapter 150 to be manipulated via a controller or the control handle 131 of the master module 110. When the surgical tools are attached to the apparatus 100, the motion and positioning of the surgical tools will be determined according to the control at the master module 110, specifically, the control handle 131 that is held and controlled by a surgeon.

A gripper of the surgical instrument 160 that is attached to the apparatus 100 may be closed or opened in response to an input received via the control handle 131. Further, the tip of the surgical instrument 160 attached to the apparatus 100 may be elevated and rotated by a surgeon via the control handle 131.

In other words, via a kinematic coupling between the master module 110 and the slave module 120, the surgical instrument 160 that is attached to the slave module 120 moves exactly in the same direction as the control handle 131 because the apparatus 100 is designed to translate the surgeon's hand movements at the master module 110 to the surgical instrument 160 at the slave module 120. Moreover, the surgeon's wrist movements are translated to the movements of the tip of the surgical instrument 160 because the same spatial relationship will be maintained between the master module 110 and the slave module 120. In particular, the kinematic link may be generally implemented mechanically without requiring any electronic implementation.

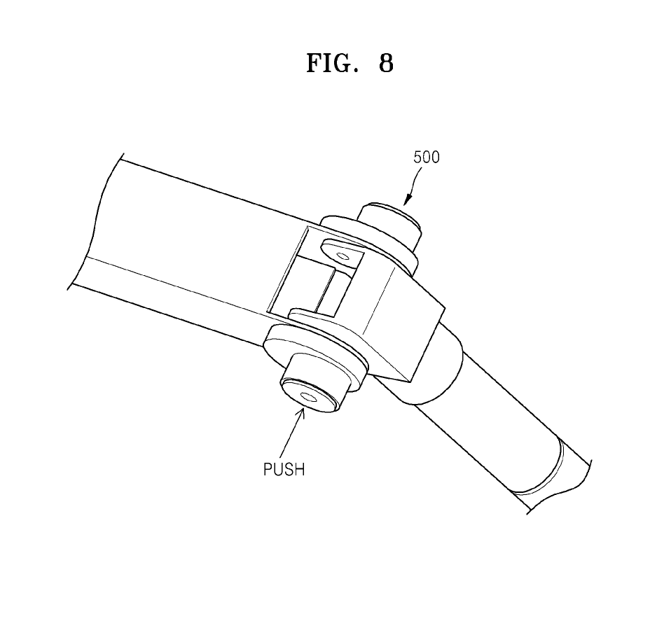

Referring to FIG. 8, the apparatus 100 may further include a brake 500 to be used with a joint 113, 123 such as a Y-joint. For example, the brake 500 may include a solenoid such as a push solenoid that is de-energized when not pushed and energized when pushed. For example, a solenoid may be provided to the joints 113, 123 shown in FIG. 1A. The brake 500 may be controlled by a brake controller 403 which is operatively connected to a power supply 402 and a pedal 401 as shown in FIG. 1D.

The brake 500 may be controlled by a user via the pedal 401 shown in FIG. 1D. When the user steps on the pedal 401, all components of the apparatus 100 may stop moving and the components may stay at the stopped positions until the brake 500 is released.

The present disclosure relates to the art and science of a mechanical manipulator for endoscopic surgery. It will be apparent to those skilled in the art that various modifications and variations can be made in the example embodiment of the present invention described above without departing from the spirit or scope of the invention. Thus, it is intended that the present invention covers the modifications and variations of various example embodiments provided they come within the scope of the appended claims and their equivalents.

What is claimed is:

1. A surgical manipulator comprising:
a master module configured to be controlled by a user;
a slave module configured to move according to movement of the master module; and
a transmission module comprising gears and configured to operably connect the master module and the slave module,
wherein the master module comprises:
an upper arm with a proximal end connected to a first end portion of the transmission module;
a lower arm with a proximal end connected to a distal end of the upper arm of the master module; and
a handle portion connected to a distal end of the lower arm and configured to receive a user input,
wherein the slave module comprises:
an upper arm with a proximal end connected to a second end portion of the transmission module;
a lower arm with a proximal end connected to a distal end of the upper arm of the slave module; and
an adapter connected to a distal end of the lower arm of the slave module and configured to receive a surgical instrument,
wherein the transmission module is configured to transmit a mechanical force to the slave module in order to cause movement of at least the upper arm of the slave module, the lower arm of the slave module, the adapter, or the surgical instrument attached to the adaptor; and
wherein the mechanical force is generated in response to the user input received via the handle portion.

2. The surgical manipulator of claim 1, wherein the transmission module is further configured to:
rotate the upper arm of the master module and the upper arm of the slave module synchronously;
raise or lower the upper arm of the slave module synchronously in response to raising or lowering of the upper arm of the master module;
move the lower arm of the slave module back and forth synchronously in response to back and forth movement of the lower arm of the master module;
raise or lower the upper and lower arms of the slave module synchronously in response to raising or lowering the upper and lower arms of the master module;
rotate the adapter of the slave module synchronously in response to rotation of the handle portion of the master module in order to rotate the lower arms of the master module and the slave module; and
move the adapter back and forth in response to back and forth movement of the handle portion.

3. The surgical manipulator of claim 2, wherein a range of rotation of the upper arm of the master module and the upper arm of the slave module is between approximately ±46 degrees.

4. The surgical manipulator of claim 2, wherein a range of a variable angle between the upper arm of the master module and the lower arm of the master module is approximately ±50 degrees.

5. The surgical manipulator of claim 2, wherein a range of motion for raising or lowering the upper arm of the master module is approximately ±30 degrees.

6. The surgical manipulator of claim 2, wherein a range of rotation of the lower arm of the master module is approximately ±170 degrees.

7. The surgical manipulator of claim 2, wherein the handle portion is rotatable about a vertical axis and movable back and forth or up and down.

8. The surgical manipulator of claim 1, wherein the adapter is detachable from the slave module and replaceable with another type of adapter.

9. The surgical manipulator of claim 8, further comprising a plurality of adapters, wherein each of the plurality of adapters is configured to receive a specific type of surgical instrument that is compatible with the corresponding adapter.

10. The surgical manipulator of claim 9, wherein the plurality of adapters are configured to receive various commercially available surgical instruments that are manufactured by different manufacturers.

11. The surgical manipulator of claim 1, wherein the transmission module further comprises a pulley.

12. The surgical manipulator of claim 1, wherein:
a length of the upper arm of the master module is approximately 10 to 15 inches;
a length of the lower arm of the master module is approximately 10 to 15 inches;
a length of the upper arm of the slave module is approximately 10 to 15 inches; and
a length of the lower arm of the slave module is approximately 10 to 15 inches.

13. The surgical manipulator of claim 1, wherein:
an angle between the upper arm and the lower arm of the master module is within a range of approximately 1 to approximately 359 degrees according to a position of the lower arm of the master module; and
an angle between the upper arm and the lower arm of the slave module is within a range of approximately 1 to approximately 359 degrees according to the position of the lower arm of the master module.

14. The surgical manipulator of claim 13, wherein the angle between the upper arm and the lower arm of the master module and the angle between the upper arm and the lower arm of the slave module are substantially the same when the upper arm and the lower arm of the master module are positioned to form an angle of approximately 90 degrees.

15. The surgical manipulator of claim 13, wherein the angle between the upper arm and the lower arm of the slave module is greater than the angle between the upper arm and the lower arm of the master module when the upper arm and the lower arm of the master module are positioned to form an angle of less than approximately 90 degrees.

16. The surgical manipulator of claim 13, wherein the angle between the upper arm and the lower arm of the slave module is less than the angle between the upper arm and the lower arm of the master module when the upper arm and the lower arm of the master module are positioned to form an angle of greater than approximately 90 degrees.

17. The surgical manipulator of claim 1, wherein the upper arm of the master module and the upper arm of the slave module are rotated about an X-axis that is a lengthwise axis of the transmission module.

18. The surgical manipulator of claim 17, wherein the lower arm of the slave module and the lower arm of the master module are moved back and forth along the X-axis.

19. The surgical manipulator of claim 18, wherein at least an angle between the lower arm and the upper arm of the slave module or an angle between the lower arm and the upper arm of the master module is changed when the lower arm of the slave module and the lower arm of the master module are moved back and forth synchronously.

20. The surgical manipulator of claim 1, wherein the transmission module requires no electrical power.

21. The surgical manipulator of claim 1, wherein each of the master module and the slave module has seven degrees of freedom.

22. The surgical manipulator of claim 1, further comprising a multi-channel breaker configured to stop motion of the master module and the slave module at a desired position.

23. The surgical manipulator of claim 22, further comprising an actuator configured to power the breaker.

24. The surgical manipulator of claim 1, wherein:
the handle portion comprises a wheel configured to cause a tip of the surgical instrument to at least rotate, bend, or grab; and
the wheel is rotatable at least up and down or left and right.

25. The surgical manipulator of claim 1, further comprising a counterweight or spring balancer configured to balance weights provided in the master module and the slave module.

26. The surgical manipulator of claim 1, wherein the adapter comprises a guide pin configured to align and stabilize attachment of the surgical instrument to the adapter.

27. The surgical manipulator of claim 1, further comprising multiple master modules and multiple slave modules, wherein each of the multiple slave modules is configured to be positioned at various heights.

28. The surgical manipulator of claim 1, wherein the adapter has three degrees of freedom.

29. The surgical manipulator of claim 1, wherein:
the upper arm and the lower arm of the master module are connected via a first joint; and
the upper arm and the lower arm of the slave module are connected via a second joint.

30. The surgical manipulator of claim 29, wherein each of the first joint and the second joint comprises a brake structure configured to hold the corresponding first joint and second joint at a desired position.

31. A surgical manipulator comprising:
a master module configured to be controlled by a user;
a slave module configured to move according to movement of the master module;
a transmission module comprising gears and configured to operably connect the master module and the slave module; and
a multi-channel breaker configured to stop motion of the master module and the slave module at a desired position,
wherein the master module comprises:
an upper arm with a proximal end connected to a first end portion of the transmission module;
a lower arm with a proximal end connected to a distal end of the upper arm of the master module; and
a handle portion connected to a distal end of the lower arm and configured to receive a user input,
wherein the slave module comprises:
an upper arm with a proximal end connected to a second end portion of the transmission module;
a lower arm with a proximal end connected to a distal end of the upper arm of the slave module; and
an adapter connected to a distal end of the lower arm of the slave module and configured to receive a surgical instrument.

32. A surgical manipulator comprising:
a plurality of master modules configured to be controlled by a user;
a plurality of slave modules configured to move according to movement of the respectively corresponding plurality of master modules and to be positioned at various heights;
a plurality of transmission modules comprising gears and configured to operably connect the plurality of master modules and the respectively corresponding plurality of slave modules; and
wherein each of the plurality of master modules comprises:
an upper arm with a proximal end connected to a first end portion of the transmission module;
a lower arm with a proximal end connected to a distal end of the upper arm of the master module; and
a handle portion connected to a distal end of the lower arm and configured to receive a user input,
wherein each of the plurality of slave modules comprises:
an upper arm with a proximal end connected to a second end portion of the transmission module;
a lower arm with a proximal end connected to a distal end of the upper arm of the slave module; and
an adapter connected to a distal end of the lower arm of the slave module and configured to receive a surgical instrument.
